(19) United States
(12) Patent Application Publication
(10) Pub. No.: US 2005/0112071 A1
(43) Pub. Date: May 26, 2005

(54) Hypoestoxides, derivatives, agonists and crude extracts thereof for use in cosmetic compositions

(75) Inventors: Emmanuel A. Ojo-Amaize, Glendora, CA (US); Emeka J. Nchekwube, Morgan Hill, CA (US); Howard B. Cottam, Escondido, CA (US); Olusola A. Oyemade, Rancho Cucamonga, CA (US)

Correspondence Address: PILLSBURY WINTHROP LLP, 725 S. FIGUEROA STREET, SUITE 2800, LOS ANGELES, CA, 90017, US

(73) Assignee: PARAQUEST, INC.

(21) Appl. No.: 10/973,872

(22) Filed: Oct. 25, 2004

Related U.S. Application Data
(60) Provisional application No. 60/513,322, filed on Oct. 23, 2003.

(51) Int. Cl. A61K 7/42; A61K 31/56; A61K 35/78; A61K 31/19

(52) U.S. Cl. 424/59; 424/774; 424/401; 514/557; 514/559; 514/167; 514/159

(57) ABSTRACT

Methods of alleviating skin conditions are provided. The methods comprise application to a host's skin of an effective amount, in a cosmetic composition or in isolation, of hypoestoxide crude extract, hypoestes rosea dried leaf powder or a hypoestoxide, derivative or agonist thereof, such that the skin irritation or condition is improved or alleviated. The methods further include application of the hypoestoxide compounds in combination with salicylic acid derivatives, hydroxy acids, retinol and its esters, retinoic acid and its derivatives, perfumes, fragrances, aftershaves, deodorants and with vitamins.

This application claims the benefit of priority under 35 U.S.C. § 119 of provisional U.S. application Ser. No. 60/513,322, filed Oct. 23, 2003, the contents of which are hereby incorporated by reference in their entirety, as if fully set forth.

### CROSS REFERENCE TO RELATED APPLICATIONS

Expressly incorporated herein by reference are: U.S. Pat. Nos. 5,801,193, 5,994,328, 6,001,871 and 6,242,484, and co-pending applications, U.S. Ser. Nos. 09/006,946; 09/007,308; 09/298,653; and PCT WO 98/46222.

### FIELD OF THE INVENTION

This invention relates to the use of diterpene compounds in cosmetic compositions. In particular, disclosed herein are compounds including hypoestoxides, derivatives and agonists thereof, along with hypoestes rosea crude extracts and hypoestes dried leaf powder for use in cosmetic compositions. The compounds are used in treating acne-prone skin, for improving the complexion, for treating hair loss, to combat the greasy appearance of the skin, to protect against harmful effects of UV light and to protect against aging and wrinkling of the skin.

### BACKGROUND OF THE INVENTION

Skin, which is the biggest organ of the human body, is composed of epidermis, dermis and subcutaneous fat. It performs various functions such as protection, temperature control, excretion and respiration. As aging progresses, these functions rapidly decline and a variety of physiological changes occur to the skin. These age-dependent physiological changes include a decrease in thickness of epidermis, dermis and subcutaneous tissue, dryness of skin resulting from moisture reduction according to the changes of lipid composition, and the occurrence of age spots, freckles, pigmentation or various skin lesions. Epidermal Growth Factor (EGF) is believed to have an excellent effect on skin injuries because it strongly promotes the proliferation of epithelial cells, endothelial cells and fibroblasts (Carpenter, G. and Cohen, S., “Epidermal growth factor”, Ann. Rev. Biochem., 48, 192-216 (1979). The active oxygen species and free radicals which can be generated by excess UV rays, air pollution, fatigue or stress in modern life, oxidize or denature the bio-materials such as proteins, nucleic acids and membrane lipids leading to the aging of the skin. To this end, many studies have been conducted on the occurrence of wrinkles, age spots or freckles, hypersensitivity reactions, the loss of skin elasticity, and pigmentation and dryness of skin (Kumar, P., and Clark, M.: “Clinical Medicine”, 3^{rd} edition, p. 147-150, 1994, Bailliere Tindall, London).

The corticosteroids are among the most widely used drugs for various skin conditions including hypersensitivity reactions. Corticosteroids primarily exert their pharmacological action by non-selectively inhibiting the function and proliferation of different classes of immune cells resulting in suppression of hypersensitivity reactions. Unfortunately the corticosteroids are associated with a number of serious side effects, including immuno-suppression, osteoporosis and skin atrophy. Thus, alternate strategies for the treatment of skin conditions are desired.

### SUMMARY OF THE INVENTION

The clinical effectiveness of the topical application of hypoestoxides in a pharmaceutical composition in combating topical inflammation in mice has been reported (Ojo-Amaize, E. A., et. al., Cell. Immunol., 209, 149-157 (2001). Because hypoestoxides have been shown to possess anti-inflammatory properties, it is anticipated that hypoestoxide-based compounds may be effective in treating many of the skin conditions discussed above.

The present invention provides methods for reducing, alleviating or preventing skin irritation and treating or preventing skin conditions in a host, such as a human, wherein the skin conditions may include dry skin, acne, blemishes, greasy appearance of the skin, wrinkling, or the harmful effects of UV radiation. The methods comprise application to the host's skin an effective amount, in a cosmetic composition or in isolation, of hypoestoxide crude extract, hypoestes rosea dried leaf powder or a compound of formula I, such that the skin irritation or condition is improved, prevented or alleviated. 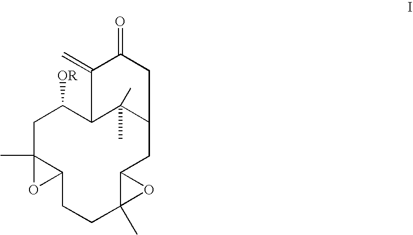
wherein R is:
a) H or acetyl,
b) P(O)(OH)₂,
c) P(O)(OH)(OM), wherein M is selected from the group consisting of an alkali metal salt and an alkaline earth metal salt,
d) P(O)OM₂ wherein M is each independently selected from the group consisting of alkali metal salts and alkaline earth metal salts,
e) Alkyl of 1 to 12 carbon atoms having 0 to 6 double bonds, said alkyl selected from the group consisting of substituted, unsubstituted, straight chain and branched alkyls,
f) (CH₂)n morpholine, wherein n=1-4,
g) morpholinomethylphenyl, ortho-aminophenyl or ortho-hydroxyphenyl,
h) (CH₂)nCOOR₂ wherein n=1-4, R₂ is each selected from the group consisting of H, an alkali metal salt, an alkaline earth metal salt, NH₄+ and N+(R₃)₄ wherein R₃ is each independently selected from the group consisting of H and an alkyl of 1 to 4 carbon atoms, or
i) COR₁ wherein R₁ is selected from the group consisting of H, (CH₂)nCH₃ wherein n=0-6, (CH₂)nCOOR₂ wherein n=1-4 and R₂ is each selected from the group consisting of H, an alkali metal salt, an alkaline earth metal salt, NH₄+ and N+(R₃)₄, and (CH₂)nN+(R₃)₄, wherein n=1-4 and R₃ is each independently selected from the group consisting of H and an alkyl of 1 to 4 carbon atoms,
   wherein an effective amount is an amount sufficient to ameliorate at least one symptom of clinical signs of skin aging, namely fine lines and wrinkles, disorganization of the “grain” of the skin, and loss of skin firmness and tonicity. An effective amount is also an amount sufficient to ameliorate at least one symptom of dry skin, acne, blemishes, greasy appearance of the skin, wrinkling, or the harmful effects of UV radiation.

### DETAILED DESCRIPTION OF THE INVENTION

As used herein, the term “host” or “subject” is taken to mean human, as well as other animals. The terms “ameliorate” or “alleviate” mean to improve, lessen the severity of or mitigate, or to remove or reduce symptoms in the humans or animals to which the compositions of this invention are administered. For example, a perceived reduction in itching constitutes alleviation, as does a reduction in rash area or fading of rash color. Reduction in histamine production as measured by any conventional assay is another example of alleviation.

Methods of treating or preventing a host's skin irritation or skin condition are provided. Skin conditions amenable to treatment by the present methods may include, but are not limited to, dry skin, acne, blemishes, greasy appearance of the skin, wrinkling, or the harmful effects of UV radiation. The present methods may also be used for treatment or prevention of the symptoms of clinical signs of skin aging, namely fine lines and wrinkles, disorganization of the “grain” of the skin, and loss of skin firmness and tonicity.

In one method, an effective amount of hypoestoxide crude extract, hypoestes rosea dried leaf powder or a compound of formula I, in a cosmetic composition or in isolation, is applied to the host's skin to alleviate or prevent irritation. Irritation may be caused by a chemical irritant, including but not limited to a cosmetic. Irritation may also be caused by a plant, including, but not limited to poison ivy, poison oak and poison sumac. Irritation may also be caused by radiation such as UV radiation or by an insect bite. 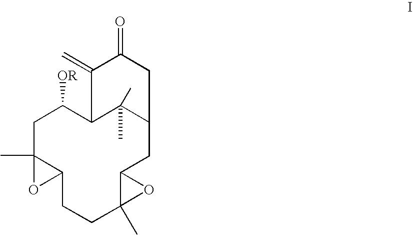
wherein R is:
a) H or acetyl,
b) P(O)(OH)₂,
c) P(O)(OH)(OM), wherein M is selected from the group consisting of an alkali metal salt and an alkaline earth metal salt,
d) P(O)OM₂ wherein M is each independently selected from the group consisting of alkali metal salts and alkaline earth metal salts,
e) Alkyl of 1 to 12 carbon atoms having 0 to 6 double bonds, said alkyl selected from the group consisting of substituted, unsubstituted, straight chain and branched alkyls,
f) (CH₂)n morpholine, wherein n=1-4,
g) morpholinomethylphenyl, ortho-aminophenyl or ortho-hydroxyphenyl,
h) (CH₂)nCOOR₂ wherein n=1-4, R₂ is each selected from the group consisting of H, an alkali metal salt, an alkaline earth metal salt, NH₄+ and N+(R₃)₄ wherein R₃ is each independently selected from the group consisting of H and an alkyl of 1 to 4 carbon atoms, or
i) COR₁ wherein R₁ is selected from the group consisting of H, (CH₂)nCH₃ wherein n=0-6, (CH₂)nCOOR₂ wherein n=1-4 and R₂ is each selected from the group consisting of H, an alkali metal salt, an alkaline earth metal salt, NH₄+ and N+(R₃)₄, and (CH₂)nN+(R₃)₄, wherein n=1-4 and R₃ is each independently selected from the group consisting of H and an alkyl of 1 to 4 carbon atoms,
   wherein an effective amount is an amount sufficient to alleviate skin irritation.

Table 1 shows the anti-irritant effect of a crude hypoestoxide extract on human subjects. Test compounds were applied on the forearms of eleven subjects between the ages of 32 and 66. The compounds were allowed to absorb before Balsam of Peru, a known irritant, was applied to the test area. Skin irritation was measured with the Minolta Chromameter, and compared with positive and negative controls. The positive control was skin treated with Balsam of Peru alone and the negative control was skin treated with Cola solution alone (10% hydro alcoholic).

TABLE 1

| Treatment | Results |
|---|---|
| 0.5% hypoestoxide crude extract | 50% reduction in irritation |
| 1.0% hypoestoxide crude extract | 60% reduction in irritation |

In another method, an effective amount of hypoestoxide crude extract, hypoestes rosea dried leaf powder or a compound of formula I, in a cosmetic composition or in isolation, is applied to the host's skin to alleviate acne. In a preferred embodiment, topical administration of 0.05% by weight hypoestoxide crude extract in a cosmetic composition was applied to five subjects with acne. Topical administration was performed twice daily for one week at the subjects' job sites. Subjects had acne vulgaris with moderate erythema with forehead and inferior mandibular distribution. The cosmetic composition consisted of the following ingredients: multi-fruit complex, cetearyl alcohol and ceteareth 20, glyceryl stearate, cetyl alcohol, tocopherol, vitamin A palmatate, allantoin, lanolin, ascorbic acid, botanical extract, xanthan gum, ceteth-20 and propylparaben. Erythema resolution, shown in Table 2, was scored on day 7 on a scale of 1 to 10, where 0=no resolution and 10=complete resolution.

TABLE 2

| Subject # | Results |
|---|---|
| 1 | 7 |
| 2 | 7 |
| 3 | 8 |
| 4 | 8 |
| 5 | 8 |

In another method, an effective amount of hypoestoxide crude extract, hypoestes rosea dried leaf powder or a compound of formula I, in a cosmetic composition or in isolation, is applied to the host's skin to alleviate psoriasis. In a preferred embodiment, 0.5% by weight of pure hypoestoxide in a cosmetic composition was applied to two subjects with psoriasis. Topical administration was performed twice daily for one week at the subjects' job sites. Subjects had crusted erythematous plaques on their elbows. The cosmetic composition consisted of the following ingredients: multi-fruit complex, cetearyl alcohol and ceteareth 20, glyceryl stearate, cetyl alcohol, tocopherol, vitamin A palmatate, allantoin, lanolin, ascorbic acid, botanical extract, xanthan gum, ceteth-20 and propylparaben. Erythema resolution was noted by observation of the subjects' plaques on day 7. Both subjects showed significant erythema resolution on day 7 in the crusted erythematous plaques.

Preferred compounds of the invention are compounds of formula I, wherein R=H and R=acetyl(hypoestoxide). The dosage of hypoestoxide crude extract, hypoestes rosea dried leaf powder or a compound of formula I in the treatment of skin conditions will vary with the progression of the condition, and with the cosmetic agent(s) or other treatments used. The number of topical treatments and the frequency will vary according to the age and response of the particular subject. In general, the total daily application of hypoestoxide crude extract, hypoestes rosea dried leaf powder or a compound of formula I varies from 1 to 4 times daily.

The hypoestoxide crude extract is an ethyl alcohol extract of hypoestes rosea dried leaves or dried leaf powder. Following extraction, the alcohol is evaporated and a paste of the extract remains behind which is the hypoestoxide crude extract.

The hypoestoxide crude extract, hypoestes rosea dried leaf powder or a compound of formula I may be used alone, or in conjunction with other cosmetic agents. Solely by way of example, hypoestoxide crude extract may be used in a cosmetic composition of the following ingredients for the treatment of acne: multi-fruit complex, cetearyl alcohol and ceteareth 20, glyceryl stearate, cetyl alcohol, tocopherol, vitamin A palmatate, allantoin, lanolin, ascorbic acid, botanical extract, xanthan gum, ceteth-20 and propylparaben.

The compounds of this invention may be used in conjunction with other treatment agents, such as salicylic acid derivatives, hydroxy acids, retinol and its esters and retinoic acid and its derivatives. The compounds of this invention may also be used in combination with perfumes, fragrances, aftershaves and deodorants and with vitamins such as vitamin D and its derivatives and vitamin B9 and its derivatives.

The percentage of the compositions and preparations may be varied and may conveniently be between about 0.01% and about 10% of the weight of a given unit formulation form. The amount of active compound in such useful compositions is such that an effective dosage level will be obtained.

All publications, patents, and patent documents are incorporated by reference herein, as though individually incorporated by reference.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

1. A method of alleviating a skin condition in a human, comprising:
applying to an area of skin afflicted with the skin condition a compound containing about 0.01% to about 10% pure hypoestoxide.

2. The method of claim 1, wherein the condition is acne.

3. The method of claim 1, wherein the condition is psoriasis.

4. The method of claim 1, wherein the condition is skin irritation.

5. The method of claim 4, wherein the skin irritation is caused by contact with an irritant selected from the group consisting of a cosmetic, a plant, poison oak, poison ivy, poison sumac, radiation, UV radiation and an insect bite.

6. The method of claim 1, wherein the condition is selected from the group consisting of wrinkling and aging.

7. The method of claim 1, wherein the compound is incorporated in a cosmetic composition.

8. The method of claim 7, wherein the cosmetic composition contains multi-fruit complex, cetearyl alcohol and ceteareth 20, glyceryl stearate, cetyl alcohol, tocopherol, vitamin A palmatate, allantoin, lanolin, ascorbic acid, botanical extract, xanthan gum, ceteth-20 and propylparaben.

9. The method of claim 1, wherein the compound is applied in combination with an agent selected from the group consisting of salicylic acid derivatives, hydroxy acids, retinol and its esters, retinoic acid and its derivatives, perfumes, fragrances, aftershaves, deodorants, vitamin D and its derivatives, and vitamin B9 and its derivatives.

10. A method of alleviating a skin condition in a human, comprising:
applying to an area of skin afflicted with the skin condition a compound containing about 0.01% to about 10% hypoestoxide crude extract or 0.01% to about 10% hypoestes rosea dried leaf powder.

11. The method of claim 10, wherein the condition is acne.

12. The method of claim 10, wherein the condition is psoriasis.

13. The method of claim 10, wherein the condition is skin irritation.

14. The method of claim 13, wherein the skin irritation is caused by contact with an irritant selected from the group consisting of a cosmetic, a plant, poison oak, poison ivy, poison sumac, radiation, UV radiation and an insect bite.

15. The method of claim 10, wherein the condition is selected from the group consisting of wrinkling and aging.

16. The method of claim 10, wherein the compound is incorporated in a cosmetic composition.

17. The method of claim 16, wherein the cosmetic composition contains multi-fruit complex, cetearyl alcohol and ceteareth 20, glyceryl stearate, cetyl alcohol, tocopherol, vitamin A palmatate, allantoin, lanolin, ascorbic acid, botanical extract, xanthan gum, ceteth-20 and propylparaben.

18. The method of claim 10, wherein the compound is applied in combination with an agent selected from the group consisting of salicylic acid derivatives, hydroxy acids, retinol and its esters, retinoic acid and its derivatives, perfumes, fragrances, aftershaves, deodorants, vitamin D and its derivatives, and vitamin B9 and its derivatives.

19. A method of alleviating a skin condition in a human, comprising:
applying to an area of skin afflicted with the skin condition a compound of formula I

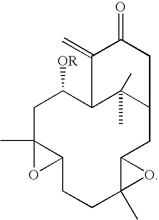

20. The method of claim 1, wherein R is selected from the group consisting of:
a) H or acetyl,
b) P(O)(OH)₂,
c) P(O)(OH)(OM), wherein M is selected from the group consisting of an alkali metal salt and an alkaline earth metal salt,
d) P(O)OM₂ wherein M is each independently selected from the group consisting of alkali metal salts and alkaline earth metal salts,
e) Alkyl of 1 to 12 carbon atoms having 0 to 6 double bonds, said alkyl selected from the group consisting of substituted, unsubstituted, straight chain and branched alkyls,
f) (CH₂)n morpholine, wherein n=1-4,
g) morpholinomethylphenyl, ortho-aminophenyl or ortho-hydroxyphenyl,
h) (CH₂)nCOOR₂ wherein n=1-4, R₂ is each selected from the group consisting of H, an alkali metal salt, an alkaline earth metal salt, NH₄+ and N+(R₃)₄ wherein R₃ is each independently selected from the group consisting of H and an alkyl of 1 to 4 carbon atoms, and
i) COR₁ wherein R₁ is selected from the group consisting of H, (CH₂)nCH₃ wherein n=0-6, (CH₂)nCOOR₂ wherein n=1-4 and R₂ is each selected from the group consisting of H, an alkali metal salt, an alkaline earth metal salt, NH₄+ and N+(R₃)₄, and (CH₂)nN+(R₃)₄, wherein n=1-4 and R₃ is each independently selected from the group consisting of H and an alkyl of 1 to 4 carbon atoms.

21. The method of claim 19, wherein the condition is acne.

22. The method of claim 19, wherein the condition is psoriasis.

23. The method of claim 19, wherein the condition is skin irritation.

24. The method of claim 23, wherein the skin irritation is caused by contact with an irritant selected from the group consisting of a cosmetic, a plant, poison oak, poison ivy, poison sumac, radiation, UV radiation and an insect bite.

25. The method of claim 19, wherein the condition is selected from the group consisting of wrinkling and aging.

26. The method of claim 19, wherein the compound is incorporated in a cosmetic composition.

27. The method of claim 26, wherein the cosmetic composition contains multi-fruit complex, cetearyl alcohol and ceteareth 20, glyceryl stearate, cetyl alcohol, tocopherol, vitamin A palmatate, allantoin, lanolin, ascorbic acid, botanical extract, xanthan gum, ceteth-20 and propylparaben.

28. The method of claim 19, wherein the compound is applied in combination with an agent selected from the group consisting of salicylic acid derivatives, hydroxy acids, retinol and its esters, retinoic acid and its derivatives, perfumes, fragrances, aftershaves, deodorants, vitamin D and its derivatives, and vitamin B9 and its derivatives.

29. A method of preventing skin irritation in a human, comprising:
applying to an area of skin afflicted with the skin irritation a diterpene compound.

30. The method of claim 29, wherein the diterpene compound is selected from the group consisting of a compound containing about 0.01% to about 10% pure hypoestoxide, a compound containing about 0.01% to about 10% hypoestoxide crude extract, a compound containing about 0.01% to about 10% hypoestes rosea dried leaf powder, and a compound of formula I 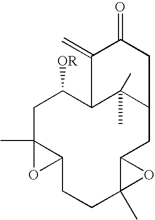
wherein R is selected from the group consisting of:
a) H or acetyl,
b) P(O)(OH)₂,
c) P(O)(OH)(OM), wherein M is selected from the group consisting of an alkali metal salt and an alkaline earth metal salt,
d) P(O)OM₂ wherein M is each independently selected from the group consisting of alkali metal salts and alkaline earth metal salts,
e) Alkyl of 1 to 12 carbon atoms having 0 to 6 double bonds, said alkyl selected from the group consisting of substituted, unsubstituted, straight chain and branched alkyls,
f) (CH₂)n morpholine, wherein n=1-4,
g) morpholinomethylphenyl, ortho-aminophenyl or ortho-hydroxyphenyl,
h) (CH₂)nCOOR₂ wherein n=1-4, R₂ is each selected from the group consisting of H, an alkali metal salt, an alkaline earth metal salt, NH₄+ and N+(R₃)₄ wherein R₃ is each independently selected from the group consisting of H and an alkyl of 1 to 4 carbon atoms, and
i) COR₁ wherein R₁ is selected from the group consisting of H, (CH₂)nCH₃ wherein n=0-6, (CH₂)nCOOR₂ wherein n=1-4 and R₂ is each selected from the group consisting of H, an alkali metal salt, an alkaline earth metal salt, NH₄+ and N+(R₃)₄, and (CH₂)nN+(R₃)₄, wherein n=1-4 and R₃ is each independently selected from the group consisting of H and an alkyl of 1 to 4 carbon atoms.

31. The method of claim 29, wherein the skin irritation is caused by contact with an irritant selected from the group consisting of a cosmetic, a plant, poison oak, poison ivy, poison sumac, radiation, UV radiation and an insect bite.

32. The method of claim 29, wherein the compound is incorporated in a cosmetic composition.

33. The method of claim 32, wherein the cosmetic composition contains multi-fruit complex, cetearyl alcohol and ceteareth 20, glyceryl stearate, cetyl alcohol, tocopherol, vitamin A palmatate, allantoin, lanolin, ascorbic acid, botanical extract, xanthan gum, ceteth-20 and propylparaben.

34. The method of claim 29, wherein the compound is applied in combination with an agent selected from the group consisting of salicylic acid derivatives, hydroxy acids, retinol and its esters, retinoic acid and its derivatives, perfumes, fragrances, aftershaves, deodorants, vitamin D and its derivatives, and vitamin B9 and its derivatives.
